(19) United States
(12) Patent Application Publication
(10) Pub. No.: US 2026/0216374 A1
(43) Pub. Date: Jul. 30, 2026

(54) POU5F1B INHIBITORS

(71) Applicant: ECOLE POLYTECHNIQUE FEDERALE DE LAUSANNE (EPFL), Lausanne (CH)

(72) Inventors: Laia SIMÓ RIUDALBAS, Lausanne (CH); Didier TRONO, Vufflens-le-Chateau (CH); Laurence GOUZI ABRAMI, Saint-Sulpice (CH); Gisou VAN DER GOOT, Trelex (CH); Sandra OFFNER, Rolle (CH)

(21) Appl. No.: 19/151,652

(22) PCT Filed: Jan. 26, 2024
(86) PCT No.: PCT/EP2024/051959

§ 371 Date: Jul. 29, 2025

(30) Foreign Application Priority Data
Jan. 30, 2023 (EP) 23154002.2

(51) Int. Cl. A61K 48/00; A61K 38/17; C07K 16/18; C12N 15/113; C12Q 1/6883; G01N 33/68

(52) U.S. Cl. CPC A61K 48/005; A61K 38/1709; C07K 16/18; C12N 15/1137; C12Q 1/6883; G01N 33/6893; C12N 2310/11; C12N 2310/14; C12N 2310/531; C12Q 2600/158

(57) ABSTRACT

The invention provides an agent modulating the expression and/or activity of i) the POU5F1B protein, a fragment or variant thereof, ii) an mRNA encoding POU5F1B, a fragment or variant thereof and/or iii) the POU5F1B gene. Preferably, the agent is for use in the treatment and/or prevention of a disease linked to the expression of POU5F1B in a subject in need thereof.

### FIELD OF THE INVENTION

The invention provides an agent modulating the expression and/or activity of i) the POU5F1B protein, a fragment or variant thereof, ii) an mRNA encoding POU5F1B, a fragment or variant thereof and/or iii) the POU5F1B gene. Preferably, the agent is for use in the treatment and/or prevention of a disease linked to the expression of POU5F1B in a subject in need thereof.

### BACKGROUND OF THE INVENTION

Overall, current cancer drugs are inefficient in close to 75% of patients, and it is increasingly recognized that the future of cancer management lies in personalized approaches, whereby therapies are selected not just based on a tumor's organ of origin but rather on a precise characterization of its molecular make-up and of the patient's genetic background.

A newly available signature for an in-depth characterization of healthy and tumoral human cells is the expression pattern of transposable elements (TEs). TEs, some 4.5 million of which can be readily identified in the human genome, may account for up to 80% of our DNA, hence are the repository of a major part of our genetic heritage. TEs have been linked to disease like cancer through different mechanisms, such as insertional activation of oncogenes and disruption of tumor suppressor genes, chromosomal rearrangements, or TE-gene chimeric transcripts that activate oncogenes.

Particular TE-gene chimeric transcripts, such as expressing POU5F1B have been found overexpressed in cancer.

Although advances in therapy over the last 20 years have led to improvements in cancer survival, additional and personalized strategies are still urgently needed.

### SUMMARY OF THE INVENTION

The present invention provides an agent modulating the expression and/or activity of i) the POU5F1B protein, a fragment or variant thereof, ii) an mRNA encoding POU5F1B, a fragment or variant thereof and/or iii) the POU5F1B gene, for use in the treatment and/or prevention of a disease linked to the expression of POU5F1B in a subject in need thereof.

Further provided is a gene delivery vector comprising a nucleic acid encoding the agent of the invention.

Further provided is a host cell comprising, or modified by the introduction of, i) the gene delivery vector of the invention, or ii) a nucleic acid of the invention.

Further provided is a pharmaceutical composition comprising a therapeutically effective amount of i) an agent for use of the invention, ii) a gene delivery vector of the invention, or iii) a host cell of the invention, and a pharmaceutically acceptable carrier and/or diluent.

Further provided is a method for diagnosing a disease linked to the expression of POU5F1B in a biological sample of a subject, the method comprising
i) detecting, directly or indirectly, the expression and/or activity of the POU5F1B protein, a fragment or variant thereof, or
ii) detecting, directly or indirectly, the presence of an mRNA encoding POU5F1B, a fragment or variant thereof,
wherein the expression and/or activity of the POU5F1B protein, a fragment or variant thereof or the presence of an mRNA encoding POU5F1B, a fragment or variant thereof indicates that the subject has or is determined to have a disease linked to the expression of POU5F1B.

Further provided is a method for treating and/or preventing a disease linked to the expression of POU5F1B in a subject in need thereof, said method comprising administering a therapeutically effective amount of i) an agent for use of any one of the invention, ii) a gene delivery vector of the invention, iii) a host cell of the invention, iv) a nucleic acid of the invention, or iv) a pharmaceutical composition of the invention.

Further provided is a method of treating and/or preventing a disease linked to the expression of POU5F1B in a subject in need thereof, comprising modifying a host cell, and reintroducing the host cell (e.g. single cell or population of cells) into the subject in need thereof.

Further provided are kits for performing a method according to the invention or for the treatment and/or prevention of a disease linked to the expression of POU5F1B.

Also provided are nucleic acids encoding an siRNA, an shRNA, an snRNA, a siRNA capable of interfering the expression of short hairpin (sh), a piRNA, or a nucleic acid including an antisense oligonucleotide (e.g. ASOs, modified ASOs such as GapmeRs, . . . ) of the invention.

### DESCRIPTION OF THE FIGURES

FIG. 1—A)—Alignment of human POU5F1 (360 aa), POU5F1B (359 aa), and gorilla, orangutan and chimpanzee POU5F1B (358, 358 and 359 aa respectively) protein sequences. POU5F1B human-specific amino acids are squared (n=5), POU5F1B-specific amino acids are dashed-squared (n=7). B)—K135E and K182T mutations in POU5F1B protein triggers its nuclear re-localization on SW480 colorectal cancer cell line. C)—K135E and K182T mutations in POU5F1B protein abrogate its DRM association in SW480 colorectal cancer cell line. D)—K135E and K182T mutations in POU5F1B protein abrogate its growth promoting action on SW480 colorectal cancer cell line.

FIG. 2—A)—POU5F1B is a ubiquitylated protein. This modification is largely suppressed by the K135E and K182T mutations. B)—Treatment of cells with the ubiquitin activating enzyme inhibitor TAK-243 triggered POU5F1B re-localization from the cytoplasm to the nucleus. C)—Using the acyl-resin-assisted capture (Acyl-RAC) method, we could demonstrate that POU5F1B is palmitoylated (+NH₂OH lane), and that this modification is severely reduced by the K135E and K182T mutations. D)—ZDHHC-17 is the palmitoyl transferase responsible for POU5F1B palmitoylation. POU5F1B dissociates from DRMs upon ZDHHC-17 knockdown.

FIG. 3—A)—POU5F1B is degraded upon capivasertib, OXA-06, and laurocapram treatment. GFP intensity is proportional to cell survival after treatment with BVdU, itself reflecting POU5F1B-DCK degradation. B) Pulse-chase analysis of POU5F1B in the presence of indicated compounds. The half-life of POU5F1B is reduced upon treatment with capivasertib, OXA-06, and Y-27632, three ROCK inhibitors, whereas the protein is stabilized by the proteasome inhibitor MG132. C) ROCK phosphorylates POU5F1B on Serine 97. Immunoprecipitation of HA-tagged POU5F1B overexpressed in SW480 cells in the presence of indicated drugs followed by Western blot with (top) HA- or (bottom)phosphoserine-specific antibodies. The wild type POU5F1B phosphoserine signal decreases upon ROCK inhibition with capivasertib, oxa-06 or Y-27632 whereas it is weaker and insensitive to these drugs with the S⁹⁷G-POU5F1B mutant.

FIG. 4—Scheme for the BVdU positive selection CRISPR/Cas9 screen to identify POU5F1B protein stabilizers.

FIG. 5—Identification as MET and CD47 as a POU5F1B protein stabilizer using CRISPR/Cas9 positive selection screening. Log-fold change (LFC) of sgRNAs on day 37 relative to day 0 of screen. Results from replicates 1 (FIG. 5A), 2 (FIG. 5 B) and 3 (FIG. 5 C) from POU5F1B-DCK* Cas9 SW480 cells are represented. Candidates (True) have a Z-score average log-fold change above 1.9. CD47 and MET are highlighted in purple and red respectively. D) Ranking of the genes whose sgRNAs were most enriched in POU5F1B-DCK* Cas9 SW480 cells on day 37 of 400 μM, 600 μM and 800 μM BVdU treatment. Gene rank in corresponding DCK* screen is included for reference.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc. For example, at least one post-translational modification means one or more post-translational modification, two or more post-translational modifications, three or more post-translational modifications, etc.,

As used herein the terms “subject”, “subject in need thereof”, or “patient”, “patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human suffering from a disease linked to the expression of POU5F1B (such as, e.g. cancer and/or cancer metastasis) or a human that might be at risk of suffering from a disease linked to the expression of POU5F1B (such as e.g. cancer and/or cancer metastasis).

As used herein, a disease linked to the expression of POU5F1B refers to a disease where the POU5F1B gene and/or the POU5F1B protein is/are overexpressed.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity, i.e., an analogue of A will base-pair with T.

As used herein, a “biological sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The term “vector”, as used herein, refers to the non-limiting group comprising a retroviral vector, a DNA vector, a plasmid, an RNA vector, an adenoviral vector, an adenovirus associated vector, a lentiviral vector, an RNA (e.g. mRNA) targeted lipid nanoparticles (LNPs), a liposome or to a nucleic acid (DNA or RNA) molecule such as a plasmid or other vehicle, which contains one or more heterologous nucleic acid sequence(s) of the invention and, preferably, is designed for transfer between different host cells. The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

The term “about,” particularly in reference to a given quantity, number or percentage, is meant to encompass deviations of plus or minus ten percent (+10). For example, about 5% encompasses any value between 4.5% to 5.5%, such as 4.5, 4.6, 4.7, 4.8, 4.9, 5, 4.1, 5.2, 5.3, 5.4, or 5.5.

As used herein, the term “variant” of one or more nucleic acid sequence or polypeptide sequence of the invention refers to biologically active derivatives of said respective sequences. In general, the term “variant” refers to molecules having a native sequence and structure with one or more additions, substitutions (generally conservative in nature) and/or deletions (e.g. splice variants), relative to the native molecule, so long as the modifications do not destroy biological activity and which are “substantially homologous” to the reference molecule or sequence. In general, the sequences of such variants are functionally, i.e. biologically, active variants and will have a high degree of sequence homology to the reference sequence, e.g., sequence homology of more than 50%, generally more than 60%-70%, even more particularly 80%-85% or more, such as at least 90% or 95% or more, when the two sequences are aligned.

As used herein, a “fragment” of one or more nucleic acid sequence or polypeptide sequence of the invention refers to a sequence containing less nucleotides or amino acids in length than the respective sequences of the invention while retaining the biological activity described herein. Preferably, this fragment contains, at least 10%, 20%, 30%, 40%, 50%, 60%, 70%, 80%, or 90% of the entire length of the reference nucleic acid sequence or polypeptide sequence.

As used herein, “Homology” refers to the percent identity between two polynucleotide or two polypeptide sequences. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers herein to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

According to the present invention, the disease is linked to the expression of POU5F1B. In some aspects, the disease is a cancer, preferably a solid or a non-solid cancer. The cancer will be selected from the non-limiting group comprising carcinoma, sarcoma, melanoma, lymphoma, and leukemia. Preferably, the cancer is selected from the group comprising Acute Lymphoblastic Leukemia (ALL), Acute Myeloid Leukemia (AML), Adrenocortical Carcinoma, Adult, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Childhood Breast Cancer, Childhood Bronchial Tumors, Burkitt Lymphoma, Carcinoid Tumor (Gastrointestinal), Childhood Carcinoid Tumors, Carcinoma of Unknown Primary, Cardiac (Heart) Tumors, Central Nervous System, Childhood Atypical Teratoid/Rhabdoid Tumor, Childhood Embryonal Tumors, Childhood Germ Cell Tumor, Primary CNS Lymphoma, Cervical Cancer, Childhood Cervical Cancer, Cholangiocarcinoma, Chordoma, Childhood, Chronic Lymphocytic Leukemia (CLL), Chronic Myelogenous Leukemia (CML), Chronic Myeloproliferative Neoplasms, Colorectal Cancer, Childhood Colorectal Cancer, Childhood Craniopharyngioma, Cutaneous T-Cell Lymphoma, Ductal Carcinoma In Situ (DCIS), Embryonal Tumors, Endometrial Cancer (Uterine Cancer), Childhood Ependymoma, Esophageal Cancer, Childhood Esophageal Cancer, Esthesio neuroblastoma, Ewing Sarcoma (Bone Cancer), Childhood Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric, (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors (Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Childhood Head and Neck Cancers, Childhood Heart Tumors, Hepatocellular (Liver) Cancer, Langerhans Cell Histiocytosis, Hodgkin Lymphoma, Hypopharyngeal Cancer (Head and Neck Cancer), Intraocular Melanoma, Childhood Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Childhood Laryngeal Cancer, Papillomatosis, Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Intraocular (Eye) Melanoma, Childhood Intraocular Melanoma, Merkel Cell Carcinoma (Skin Cancer), Malignant Mesothelioma, Childhood Mesothelioma, Metastatic Squamous Neck Cancer with Occult Primary (Head and Neck Cancer), Midline Tract Carcinoma Involving NUT Gene, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplasia Syndromes, Myelodysplastic/Myeloproliferative Neoplasms, Myelogenous Leukemia, Chronic (CML), Myeloid Leukemia, Acute (AML), Chronic Myeloproliferative Neoplasms, Nasal Cavity and Paranasal Sinus Cancer (Head and Neck Cancer), Nasopharyngeal Cancer (Head and Neck Cancer), Childhood Nasopharyngeal Cancer, Neuroblastoma, Non-Hodgkin Lymphoma (NHL) such as diffuse large B cell lymphoma, Non-Small Cell Lung Cancer, Oral Cancer, Lip and Oral Cavity Cancer and Oropharyngeal Cancer (Head and Neck Cancer), Childhood Oral Cavity Cancer, Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma (Soft Tissue Sarcoma), Salivary Gland Cancer (Head and Neck Cancer), Childhood Salivary Gland Tumors, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, Childhood Stomach (Gastric) Cancer, T-Cell Lymphoma, Cutaneous, Testicular Cancer, Childhood Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Childhood Thyroid Tumors, Transitional Cell Cancer of the Renal Pelvis and Ureter (Kidney (Renal Cell) Cancer), Carcinoma of Unknown Primary, Childhood Cancer of Unknown Primary, Unusual Cancers of Childhood, Ureter and Renal Pelvis, Transitional Cell Cancer (Kidney (Renal Cell) Cancer, Urethral Cancer, Endometrial Uterine Cancer, Uterine Sarcoma, Vaginal Cancer, Childhood Vaginal Cancer, Vascular Tumors (Soft Tissue Sarcoma), Vulvar Cancer, and Wilms Tumor or a combination of one of more of these cancers.

Preferably, the cancer is a solid tumor cancer. More preferably, the solid tumor cancer is selected from the group comprising bladder cancer (BLCA), breast cancer (BRCA), colorectal cancer (COAD and READ), cervical squamous cell carcinoma and endocervical adenocarcinoma (CESC), uterine corpus endometrial carcinoma (UCEC), ovarian cancer (OV), esophageal cancer (ESCA), stomach cancer (STAD), kidney cancer (KICH, KIRC, and KIRP), lung squamous cell carcinoma (LUSC), lung adenocarcinoma (LUAD), prostate cancer (PRAD), thyroid carcinoma (THCA), thymoma (THYM), or a combination of one of more thereof.

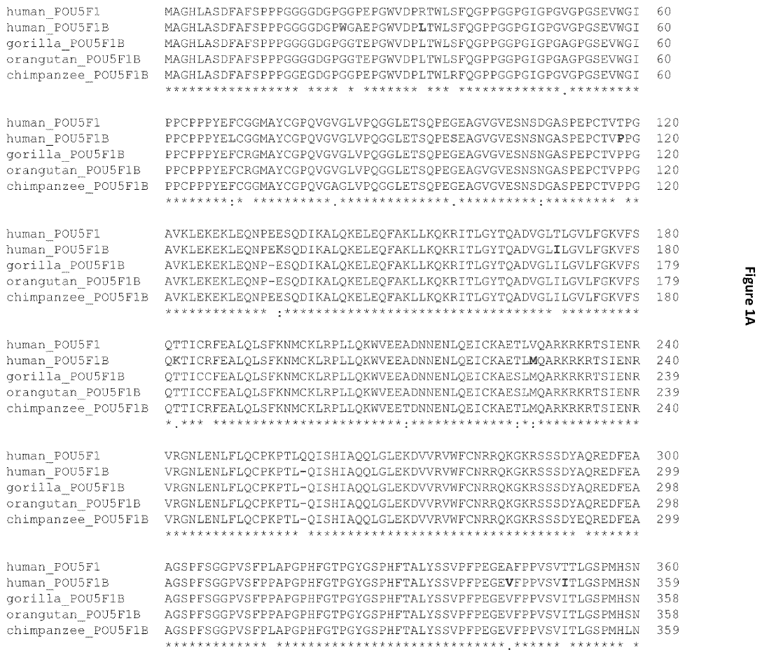

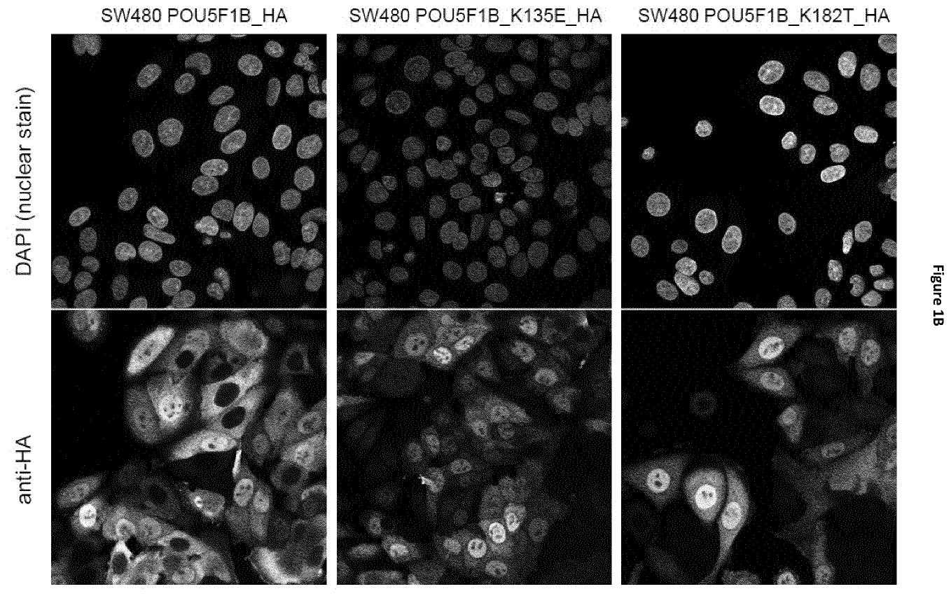

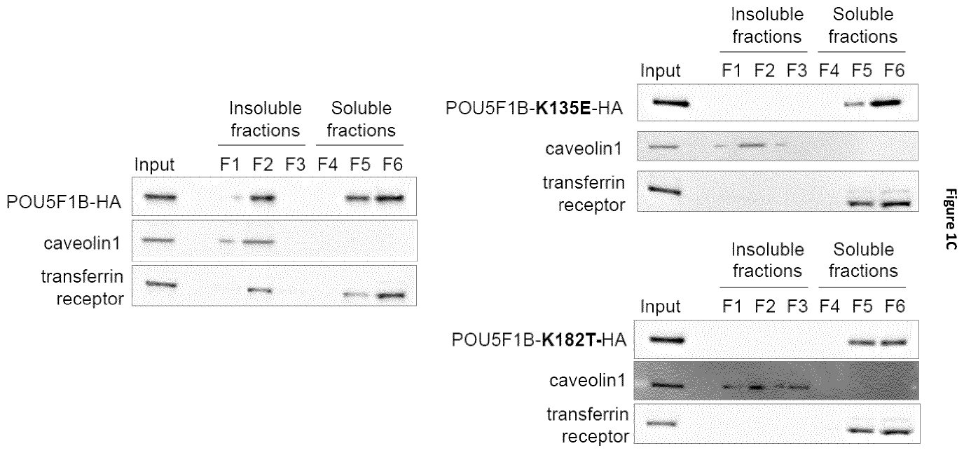

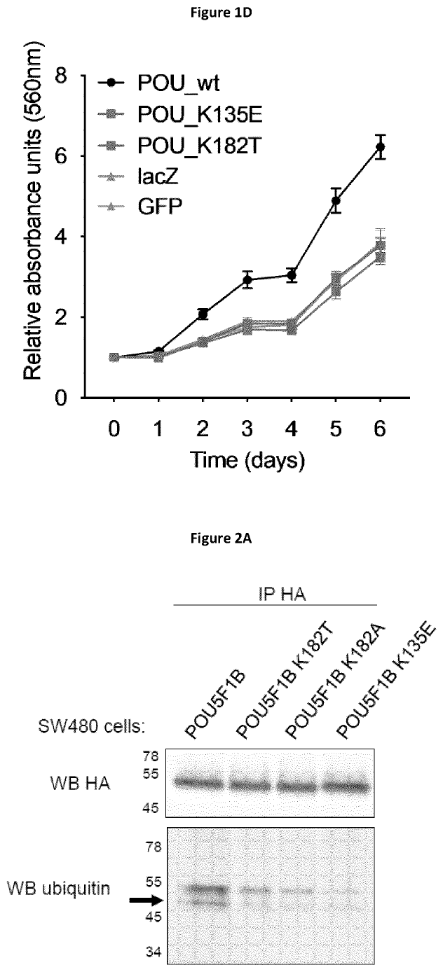

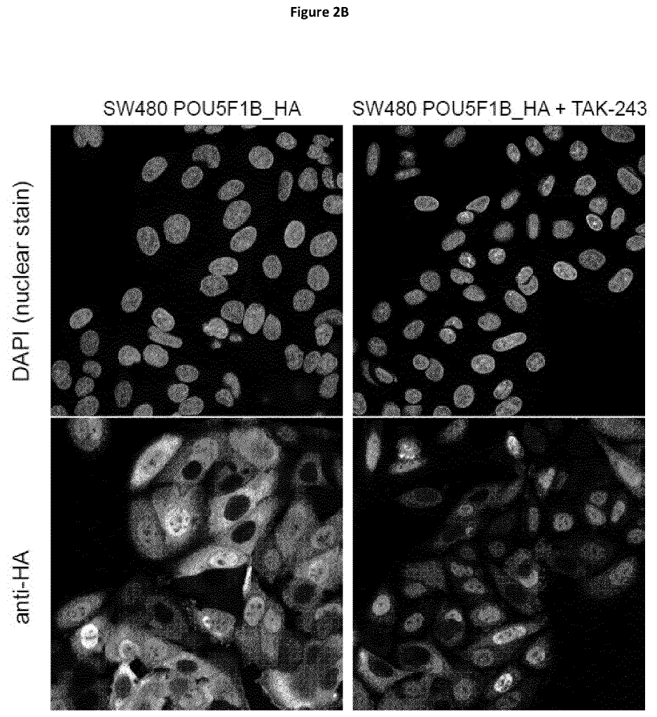

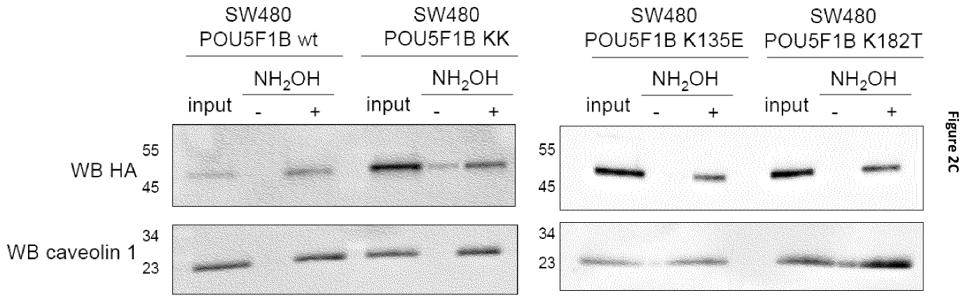

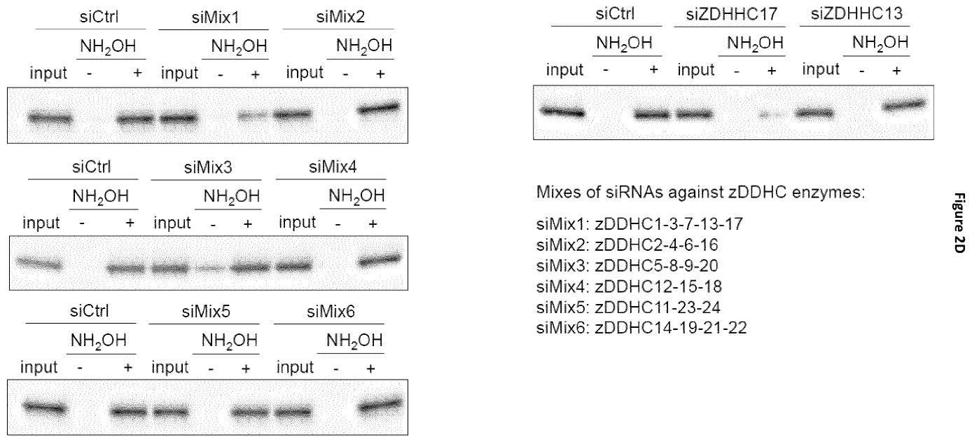

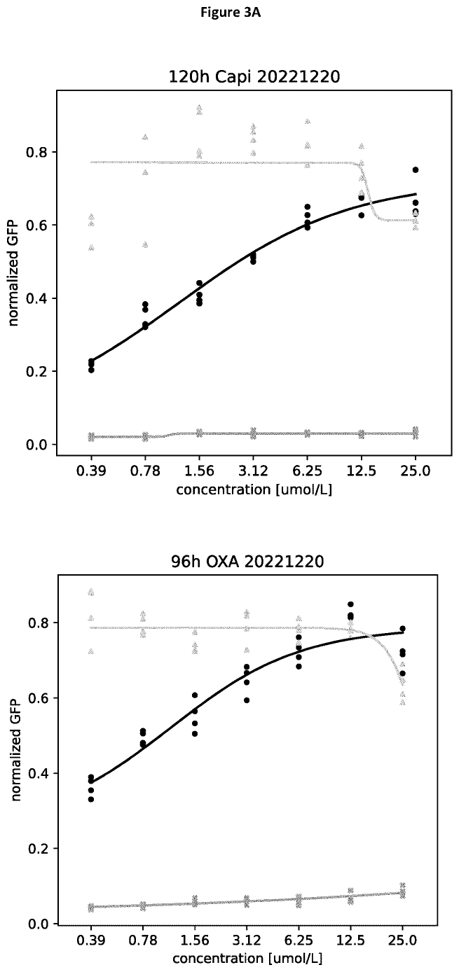

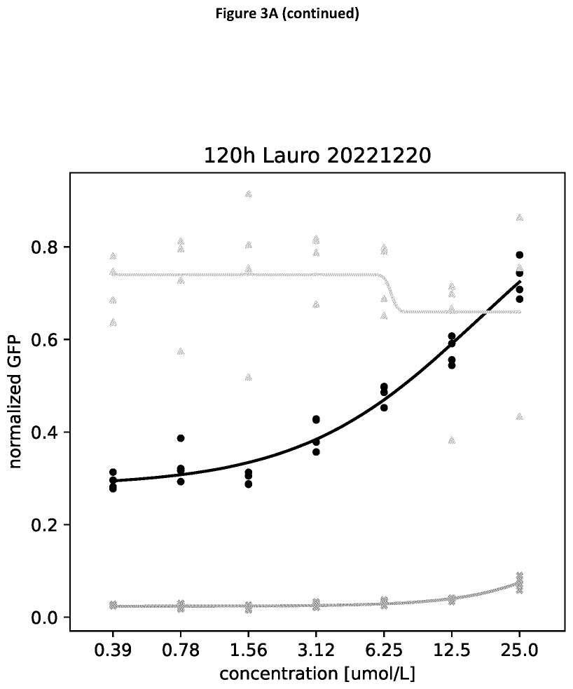

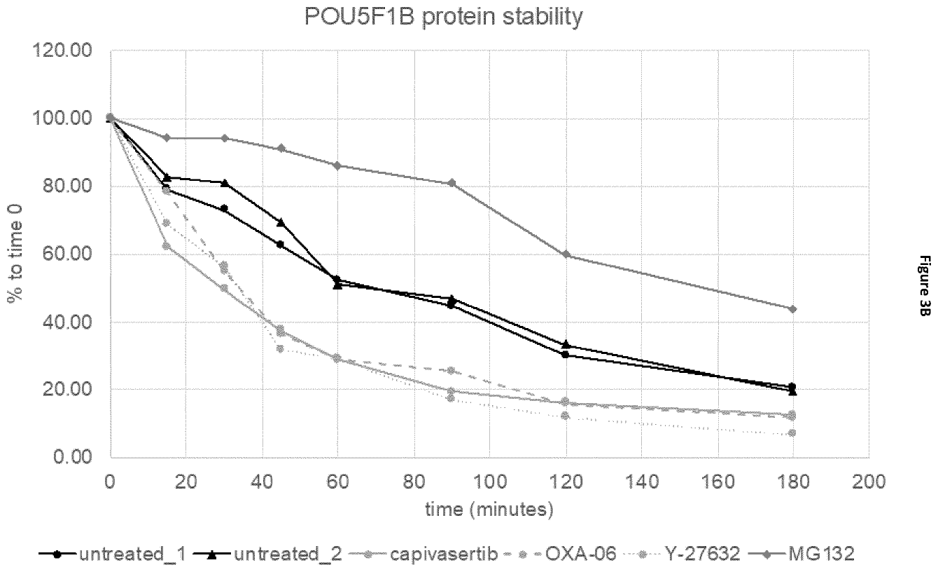

The term “POU5F1B” refers to a coding sequence and its protein product. The POU5F1B retrogene is transcribed by activation of upstream transposable element-embedded regulatory sequences in several human tumors including colon, stomach, breast, prostate and uterus. Its expression is a negative prognostic factor in colorectal and gastric malignancies and is associated with advanced histological grades of cervical cancer, where the POU5F1B locus is a documented papillomavirus integration hotspot. Supporting its role as a cancer-promoting factor, the POU5F1B protein stimulates the proliferation of colorectal, gastric and hepatocellular carcinoma cells in vitro and increases the growth and metastatic potential of human gastrointestinal tumor cells in mouse xenotransplantation experiments. While its relative POU5F1/OCT4 is a DNA-binding transcription factor concentrated in the nucleus, POU5F1B accumulates in the cytoplasm where it associates with detergent-resistant membrane (DRM) subdomains. The two proteins differ at 15 out of 359/360 positions, 5 of which are unique to human POU5F1B compared with its orthologues from other great apes (FIG. 1A).

While focusing on the role of POU5F1B, the Inventors surprisingly discovered that the action of POU5F1B, in particular its oncogenic action, requires ubiquitylation of lysine residues found only in the human protein, and that this post-translational modification is essential for POU5F1B to localize to the cytoplasm. They also determined that POU5F1B is then acylated by the ZDHHC17 palmitoyl-transferase, which leads to its association with detergent-resistant membrane subdomains where it recruits adhesion molecules such as integrin-b1, resides in proximity with Rab11a/b and fosters the formation of focal adhesions. They finally uncovered that POU5F1B is phosphorylated on Serine 97 by the ROCK kinases, that this modification too is essential for POU5F1B oncogenic action, and that it can be abrogated by treatment with ROCK inhibitors.

In one aspect, the present invention provides an agent modulating the expression and/or activity of i) the POU5F1B protein, a fragment or variant thereof, ii) an mRNA encoding POU5F1B, a fragment or variant thereof and/or iii) the POU5F1B gene. Preferably, the agent is for use in the treatment and/or prevention of a disease linked to the expression of POU5F1B in a subject in need thereof.

The agent is usually selected from the group comprising a chemical agent, an oligonucleotide, an antibody or an antigen-binding fragment thereof, a peptide, or a combination of one or more thereof.

Where the agent is a nucleic acid, it will be selected from the group comprising an siRNA, an shRNA, a piRNA, an snRNA, an siRNA capable of interfering the expression of short hairpin (sh), a guide RNA, a nucleic acid including an antisense oligonucleotide or a combination of one or more thereof.

The terms “siRNA” and “short interfering RNA” as used herein are interchangeable and refer to single-stranded or double-stranded RNA molecules that are capable of inducing RNA interference. SiRNA molecules typically have a duplex region that is between 18 and 30 base pairs in length. Exemplary siRNA of the invention may, e.g. be selected from the group non-limiting group comprising siRNAs silencing the ZDHHC gene product, a MET gene product, a CD47 gene product or the POU5F1B gene product, or any other enzyme involved in the ubiquitylation, palmitoylation or phosphorylation of the POU5F1B protein.

The terms “piRNA” and “Piwi-interacting RNA” are interchangeable and refer to a class of small RNAs involved in gene silencing. PiRNA molecules typically are between 26 and 31 nucleotides in length.

The term “shRNA” as used herein refers to a nucleic acid molecule comprising at least two complementary portions hybridized or capable of specifically hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. Exemplary shRNA of the invention may, e.g. be selected from the non-limiting group comprising i) a shRNA designed to be uniquely and selectively recognizing the 5′ untranslated region of POU5F1B mRNA such as e.g. shRNA1_POU5F1B (SEQ ID No. 5) and the shRNA2_POU5F1B (SEQ ID No. 6), or a sequence sharing at least 80%, 85%, 90%, 95%, 98% or 99% sequence identity therefrom,
ii) a shRNA designed to be uniquely and selectively recognizing a ZDHHC gene product such as e.g. siRNA_ZDHHC17 (SEQ ID No. 7) or a sequence sharing at least 80%, 85%, 90%, 95%, 98% or 99% sequence identity therefrom, iii) a shRNA designed to be uniquely and selectively recognizing any other enzyme involved in the ubiquitylation, palmitoylation or phosphorylation of the POU5F1B protein, and iv) a shRNA designed to be uniquely and selectively recognizing a MET gene product or a CD47 gene product.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more acid nucleic sequence of the invention, preferably a POU5F1B gene product, a ZDHHC gene product, a MET gene product, a CD47 gene product or any other enzyme involved in the ubiquitylation, palmitoylation or phosphorylation of the POU5F1B protein.

The terms “guide RNA” and “gRNA” are interchangeable and refer to a class of RNAs that specifically recognize the target DNA region of interest and directs nuclease (e.g. Cas nuclease) there for editing. The gRNA is made up of two parts: crispr RNA (crRNA), a 17-21 e.g. 17, 18, 19, 20, or 21 nucleotide sequence complementary to the target DNA, and a tracr RNA, which serves as a binding scaffold for the nuclease. The gRNA of the invention is typically designed to be uniquely and selectively targeting i) any sequence of the whole genomic region encompassing non-coding regulatory elements and coding regions for POU5F1B gene (GRCh37/hg19) chr8: chr8: 128,256,00-128,432,000 or ii) any sequence of the whole genomic region encompassing non-coding regulatory elements and coding regions of one or more genes that encode a transcript or a protein interacting with POU5F1B.

In one specific aspect, the gRNA of the invention is typically designed to be uniquely and selectively targeting i) (and replacing) one or more sites containing nucleotides encoding the amino acids that will be the subject of post-translational modifications of POU5F1B gene, as described herein, ii) one or more transposable elements that serve as promoter of the POU5F1B gene, iii) one or more enhancer of the POU5F1B gene or iv) one or more flanking regions of the POU5F1B gene. In an aspect of the invention, the guide RNA is a single guide RNA (sgRNA).

Examples of transposable elements (TEs) that serve as promoter of the POU5F1B gene are selected from the group comprising AluJr4, HAL1, AluSg7, LIPA16, AluSx, LIPA8, LIPA5, LIPA8, LIPA7, AluJr, LIPA7, MIR, L2a, L2b, L2c, MIRb, LTR16A, THEID, MLT2B4, ERVL-B4-int, LTR12, MIR3, LIPA3, LIPA2, L2, LTR33, DCP2, YIPF4, LTR66 and L2d2, a variant, a fragment or a combination thereof. These TEs are known in the art and are described, e.g. in WO/2020/141213 (EPFL).

Exemplary gRNA of the invention targeting one or more transposable elements that serve as promoter of the POU5F1B gene may, e.g. be selected from the non-limiting group comprising:
LTR66-g1 5′-TCACATCATTCTCACCACTCTGG-3′ (SEQ ID No. 10) and
LTR66-g2 5′-GGAGCAGTCTCCTGAAGCTTTGG-3′ (SEQ ID No. 11), or a sequence sharing at least 80%, 85%, 90%, 95%, 98% or 99% sequence identity therefrom.

Exemplary gRNA of the invention targeting one or more enhancer (e.g. hg19 chr8: 128,409,691-128,415, 139, and hg19 chr8: 128,402, 759-128,405,287) of the POU5F1B gene may, e.g. be selected from the non-limiting group comprising:
enh-g1 5′-GACGATGAGGGTATTAACTCTGG-3′ (SEQ ID No. 12) and
enh-g2 5′-GGTAATATGTTTGGGCCTGTAGG-3′ (SEQ ID No. 13), or a sequence sharing at least 80%, 85%, 90%, 95%, 98% or 99% sequence identity therefrom.

Exemplary gRNA of the invention targeting one or more flanking regions of the POU5F1B gene may, e.g. be selected from the non-limiting group comprising:
POU5F1B_g1 5′-CTAGGTCATAAAGAACGCAG-3′ (SEQ ID No. 14) and
POU5F1B_g2 5′-ATGCCGTTAGCGGTCAAAAG-3′ (SEQ ID No. 15), or a sequence sharing at least 80%, 85%, 90%, 95%, 98% or 99% sequence identity therefrom.

Exemplary gRNA of the invention targeting one or more genes that encode a protein interacting with POU5F1B (such as e.g. MET or CD47), will be selected from the non-limiting group comprising:

| single_guide_RNA_ | Gene | SEQ |
|---|---|---|
| sequence (5′>3′) | Symbol | ID No. |
| AGCTGTGGCAGCGTCAACAG | MET | 17 |
| CCGATCGCACACATTTGTCG | MET | 18 |
| CTCACTGATATCGAATGCAA | MET | 19 |
| TACTGTATTGTGTTGTCCCG | MET | 20 |
| TGAGATCAAAGTATTTGGAA | MET | 21 |
| TGATGTCCCAAGATTAGCTA | MET | 22 |
| TGCGTATGTCAGCAAGCCTG | MET | 23 |
| ACAGGAGTATAGCAAAAATT | CD47 | 24 |
| AGTGATGCTGTCTCACACAC | CD47 | 25 |
| ATCGAGCTAAAATATCGTGT | CD47 | 26 |
| CTACTGAAGTATACGTAAAG | CD47 | 27 |
| CTTACCTGGGACGAAAAGAA | CD47 | 28 |
| GCACTTAAATATAGATCCGG | CD47 | 29 |
| TTGCACTACTAAAGTCAGTG | CD47 | 30 |

or a sequence sharing at least 80%, 85%, 90%, 95%, 98% or 99% sequence identity therefrom.

Non-limiting examples of modified ASOs include the GapmeRs. As used herein, a GapmeR is a chimeric antisense oligonucleotide that contains a central block of deoxynucleotide monomers sufficiently long to induce RNase H cleavage. Usually, the GapmeRs of the invention are directed against one or more nucleic acid sequences of the invention, preferably a POU5F1B gene product, a ZDHHC gene product, a MET gene product, a CD47 gene product or any other enzyme involved in the ubiquitylation, palmitoylation or phosphorylation of the POU5F1B protein. Modified ASOs such as modified GapmeRs are also encompassed in the present invention and comprise, e.g. GapmeRs with fixed chemical modification architectures selected from the group comprising i) a gapmer with five 2′-O-methoxyethyl (MOE) modifications in each flank, and a central gap of 10 unmodified dans (e.g. 5-10-5 MOE design), and ii) a gapmer employing three or four locked nucleic acid (LNA) modifications in each flank (e.g. 3-10-3 or 4-8-4 LNA designs), as well as a combination of one or more thereof.

In one aspect, the agent of the invention modulates the expression and/or activity of the POU5F1B gene by using a gene editing system such as, e.g. the CRISPR-based gain/loss-of-function system. Usually, the CRISPR-based gain/loss-of-function system comprises at least one single guide RNA (sgRNA), or crRNA and tracrRNA, as described herein and a structure-guided endonuclease such as an RNA-guided endonuclease.

Any suitable naturally occurring, or engineered, RNA-guided endonuclease can be employed as long as it is effective for binding a target DNA and it may be selected from the non-limiting group comprising Cas9, Cas12, Cpfl, and FEN-1. Preferably, the RNA-guided endonuclease is Cas9.

The CRISPR/Cas9 system has become a remarkably flexible tool for genome manipulation over the years. A unique feature of Cas9 endonuclease is its ability to bind target DNA independently of its ability to cleave target DNA.

Within the context of this disclosure, the Cas9 endonuclease is preferably a modified Cas9 endonuclease such as, e.g. an enzymatically dead Cas9. Specifically, both RuvC- and/or HNH-nuclease domains can be rendered inactive by point mutations (e.g. D10A and H840A in SpCas9), resulting in a nuclease dead Cas9 molecule that cannot cleave target DNA. However, the dead Cas9 molecule retains the ability to bind to target DNA based on the sgRNA targeting sequence, which sgRNA sequence is comprised in CRISPR-based gain/loss-of-function system.

In one aspect, the enzymatically dead Cas9 is tagged with one or more transcriptional repressor (see Andriy Didovyk, Bartomiej Borek, Lev Tsimring, and Jeff Hasty. Curr Opin Biotechnol. 2016 August; 40:177-184 which is incorporated herein by reference).

In another aspect, the enzymatically dead Cas9 is tagged with one or more epitope that is/are recognized by one or more antibody-activator/repressor effector. This enzymatically tagged dead Cas9 can then target the POU5F1B gene of the invention.

As used herein, the term “target DNA” refers to the POU5F1B gene of the invention as disclosed above, to a regulatory sequence that controls the transcription of the gene encoding the POU5F1B of the invention, or any sequence of the whole genomic region encompassing non-coding regulatory elements and coding regions of one or more genes that encode a transcript or a protein interacting with POU5F1B such as e.g. such as e.g. MET or CD47.

Preferably, the agent of the invention modulates the expression and/or activity of the POU5F1B protein, a fragment or variant thereof i) by inhibiting or impairing, directly or indirectly, at least one post-translational modification of the POU5F1B protein, fragment or variant thereof, ii) by inducing selective intracellular proteolysis of the POU5F1B protein, fragment or variant thereof or iii) by inhibiting or impairing, directly or indirectly, the stability of the POU5F1B protein, fragment or variant thereof.

In one aspect, the agent of the invention inhibits or impairs, directly or indirectly, at least one post-translational modification selected from the group comprising ubiquitylation, palmitoylation or phosphorylation of the POU5F1B protein, fragment or variant thereof.

As used herein, the term “directly” refers to the fact that the agent of the invention directly interacts with the POU5F1B protein, fragment or variant thereof or with the mRNA encoding POU5F1B to modulate their expression and/or activity.

As used herein, the term “indirectly” refers to the fact that the agent of the invention interacts with or targets one or more enzymes (e.g. E1 ubiquitin activating enzyme, E3 ubiquitin ligase enzyme, a palmitoyltransferase or a kinase) responsible of the post-translational modification(s) of the POU5F1B protein, fragment or variant thereof.

Usually, the ubiquitylation occurs on lysin residues at positions 135 and/or 182 of the POU5F1B protein.

In one aspect, the agent of the invention inhibits the ubiquitylation of POU5F1B, for instance on lysin residues at positions 135 and/or 182.

As shown in the examples, the palmitoyltransferase responsible of the palmitoylation of the POU5F1B protein, fragment or variant thereof is ZDHHC-17 that is an enzyme that contains a DHHC domain that in humans is encoded by the ZDHHC17 gene. Preferably, the agent of the invention will target this enzyme, directly or indirectly.

Examples of E1 ubiquitin activating enzymes of the invention are selected from the group comprising UBA1, UBA2, UBA3, UBA5, UBA6, UBA7, ATG7, NAE1, and SAE1 or a combination or one or more thereof.

Examples of E3 ubiquitin ligase enzymes of the invention are selected from the group comprising UBR5, RNF213, and TRIM21 or a combination or one or more thereof.

In one aspect, the agent of the invention induces selective intracellular proteolysis of the POU5F1B protein, fragment or variant thereof.

In one aspect, the agent of the invention inhibits the phosphorylation of POU5F1B, for instance on a serine residue at position 97. In one aspect, this is achieved by blocking a kinase. In one aspect, the kinase is ROCK (Rho-associated kinase) and the inhibiting compounds comprise capivasertib, OXA-06 and Y-27632.

In one aspect, the agent of the invention inhibits or impairs, directly or indirectly, the stability of the POU5F1B protein, fragment or variant thereof.

Preferably, the agent inhibiting or impairing, the stability of the POU5F1B protein, fragment or variant thereof targets MET or CD47, or a combination thereof.

As used herein, Mesenchymal Epithelial Transition (“MET”) refers to a receptor tyrosine kinase that transduces signals from the extracellular matrix into the cytoplasm by binding to hepatocyte growth factor/HGF ligand. Regulates many physiological processes including proliferation, scattering, morphogenesis and survival. Ligand binding at the cell surface induces autophosphorylation of MET on its intracellular domain that provides docking sites for downstream signaling molecules. Following activation by ligand, interacts with the PI3-kinase subunit PIK3R1, PLCG1, SRC, GRB2, STAT3 or the adapter GAB1. Recruitment of these downstream effectors by MET leads to the activation of several signaling cascades including the RAS-ERK, PI3 kinase-AKT, or PLCgamma-PKC (https://www.uniprot.org/uniprotkb/P08581/entry).

As used herein, CD47 refers to an adhesive protein that mediates cell-to-cell interactions. Involved in signal transduction, cardiovascular homeostasis, inflammation, apoptosis, angiogenesis, cellular self-renewal, and immunoregulation. Receptor for SIRPA, binding to which prevents maturation of immature dendritic cells and inhibits cytokine production by mature dendritic cells (Latour S, Tanaka H, Demeure C, Mateo V, Rubio M, Brown E J, Maliszewski C, Lindberg F P, Oldenborg A, Ullrich A, Delespesse G, Sarfati M. Bidirectional negative regulation of human T and dendritic cells by CD47 and its cognate receptor signal-regulator protein-alpha: down-regulation of IL-12 responsiveness and inhibition of dendritic cell activation. J Immunol. 2001 Sep. 1; 167 (5): 2547-54.).

In some aspects, the agent of the invention is an antibody or an antigen-binding fragment thereof.

As used herein, an “antibody” is a protein molecule that reacts with a specific antigenic determinant or epitope and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to at least one peptide of the invention. Preferably the antibody is a monoclonal antibody. Preferably also the monoclonal antibody will be selected from the group comprising the IgG1, IgG2, IgG2a, IgG2b, IgG3 and IgG4 or a combination thereof. Most preferably, the monoclonal antibody is selected from the group comprising the IgG1, IgG2, IgG2a, and IgG2b, or a combination thereof.

An “antigen binding fragment” comprises a portion of a full-length antibody. Examples of antigen binding fragments include Fab, Fab′, F(ab′)2, and Fv fragments; diabodies; linear antibodies; single-chain antibody molecules; and multispecific antibodies formed from antibody fragments.

The antibody or antigen-binding fragment thereof of the invention is typically designed to be uniquely and selectively recognizing and binding the POU5F1B protein, a fragment or variant thereof, or any enzyme involved in the ubiquitylation, palmitoylation or phosphorylation of the POU5F1B protein.

As used herein, a “chemical agent” is a compound that produces change by virtue of its chemical composition and its effects on living tissues and organisms. The chemical agent may be a small molecule inhibitor (SMI), most preferably a non-peptidyl SMI, that is preferably
i) inducing selective intracellular proteolysis of a POU5F1B protein, fragment or variant thereof,
ii) inhibiting or impairing, directly or indirectly, at least one post-translational modification selected from the group comprising ubiquitylation, palmitoylation or phosphorylation of the POU5F1B protein, fragment or variant thereof, or
iii) inhibiting or impairing, directly or indirectly, the stability of the POU5F1B protein, fragment or variant thereof.

In one aspect, the chemical agent targets an E1 ubiquitin activating enzyme, an E3 ubiquitin ligase, a palmitoyltransferase or a kinase.

Examples of chemical agents inducing selective intracellular proteolysis comprise proteolysis targeting chimera (PROTAC) protein degraders and small-molecule chemical modulators of deubiquitinating enzymes upstream of or on the proteasome. As known in the art, PROTAC is a heterobifunctional small molecule composed of two active domains and a linker capable of removing specific unwanted proteins.

An example of a chemical agent targeting an E1 ubiquitin activating enzyme comprises TAK-243.

Examples of chemical agents targeting a palmitoyltransferase such as ZDHHC-17 and comprise palmitoylation inhibitor 2-bromopalmitate.

Examples of chemical agents targeting a kinase, such as a ROCK kinase, are selected from the group comprising AZD5363, OXA-06, and Y-27632 or a combination of one or more thereof.

The present invention also contemplates a gene delivery vector, preferably in the form of a plasmid or a vector, that comprises one or more nucleic acid(s) encoding an agent of the invention. Preferably, said agent is a nucleic acid selected from the group comprising an siRNA, an shRNA, an snRNA, a piRNA, an siRNA capable of interfering the expression of short hairpin (sh), a guide RNA, and a nucleic acid including an antisense oligonucleotide or a combination of one or more thereof. Most preferably, the nucleic acid, including an antisense oligonucleotide, is selected from the group comprising an siRNA and an shRNA.

As used herein, a “vector” is capable of transferring nucleic acid sequences to target cells (e.g., viral vectors, non-viral vectors, particulate carriers, and liposomes).

Suitable vectors include derivatives of SV40 and known bacterial plasmids, e.g., E. coli plasmids col El, pCRl, pBR322, pMB9 and their derivatives, plasmids such as RP4; phage DNAs, e.g., the numerous derivatives of phage X, e.g., NM989, and other phage DNA, e.g., Ml 3 and filamentous single stranded phage DNA; yeast plasmids such as the 2 u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Various viral vectors are used for delivering nucleic acid to cells in vitro or in vivo. Non-limiting examples are vectors based on Herpes Viruses, Pox-viruses, Adeno-associated virus, Lentivirus, and others. In principle, all of them are suited to deliver an expression cassette comprising an expressible nucleic acid molecule that codes for an agent of the invention. In a preferred aspect, said viral vector is an adenoviral vector, preferably a replication competent adenovirus.

It will be appreciated that in the present method the modification following the introduction of the gene delivery vector (plasmid or vector), or the one or more nucleic acid(s) encoding the agent of the invention, to the host cell may occur ex vivo or in vitro, for instance in a cell culture and in some instances not in vivo. In other aspects, it may occur in vivo. The host cell(s) (e.g. single cell or population of cells) of the invention is then reintroduced into the patient in need thereof by any route of administration and/or delivery methods known in the art, as described herein.

The present invention further contemplates a host cell, or population of host cells, comprising, or modified by the introduction of, a gene delivery vector of the invention or a nucleic acid of the invention. Host cells can be either eukaryotic or prokaryotic cells. In one aspect, the cell is a mammalian cell, whether an autologous or an allogeneic cell.

Also contemplated in the present invention is a pharmaceutical composition comprising a therapeutically effective amount of i) an agent for use of the invention, ii) a gene delivery vector of the invention, iii) a nucleic acid of the invention, or iv) a host cell of the invention, and a pharmaceutically acceptable carrier and/or diluent.

Usually, the pharmaceutical composition of the invention is for use in the treatment and/or prevention of a disease linked to the expression of POU5F1B.

The term “therapeutically effective amount” as used herein means an amount of an agent of the invention, ii) a gene delivery vector of the invention, or iii) a host cell of the invention, high enough to significantly positively modify the symptoms and/or condition to be treated, but low enough to avoid serious side effects (at a reasonable risk/benefit ratio), within the scope of sound medical judgment. The therapeutically effective amount of an agent of the invention, ii) a gene delivery vector of the invention, or iii) a host cell of the invention is selected in accordance with a variety of factors including type, species, age, weight, sex and medical condition of the patient; the severity of the condition to be treated; the route of administration; the renal and hepatic function of the patient.

“Pharmaceutically acceptable carrier and/or diluent” means a carrier and/or diluent that is useful in preparing a pharmaceutical composition that is generally safe, non-toxic, and desirable, and includes carriers or diluents that are acceptable for human pharmaceutical use. Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable excipients include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

The pharmaceutical composition can also further comprise or provide, when the disease is cancer, at least one additional anticancer agent or therapy selected from the group comprising radiotherapy, chemotherapy, immunotherapy and hormone therapy, or a combination of one of more thereof.

The pharmaceutical composition comprising a therapeutically effective amount of a host cell and a pharmaceutically acceptable carrier and/or diluent can be administered to the subject in need thereof by any method and route known in the art and described herein.

Further contemplated is a method of diagnosing a disease linked to the expression of POU5F1B in a biological sample of a subject, the method comprising
i) detecting, directly or indirectly, the expression and/or activity of the POU5F1B protein, a fragment or variant thereof, or
ii) detecting, directly or indirectly, the presence and/or the level of transcription of an mRNA encoding POU5F1B, a fragment or variant thereof,
wherein the expression and/or activity of the POU5F1B protein, a fragment or variant thereof or the presence of an mRNA encoding POU5F1B, a fragment or variant thereof indicates that the subject has or is determined to have a disease linked to the expression of POU5F1B.

In one aspect, a differential level (e.g. upregulated) of transcription and/or expression and/or activity of POU5F1B protein, mRNA encoding said POU5F1B, a fragment or variant thereof, in said biological sample, relative to the level of corresponding said mRNA encoding POU5F1B or POU5F1B protein in a control biological sample of a disease-free subject, is indicative of the subject having a disease linked to the expression of POU5F1B.

In one aspect, the method further comprises administering a therapeutically effective amount of i) an agent of the invention, ii) a gene delivery vector of the invention, iii) a host cell of the invention, or iv) a pharmaceutical composition of the invention, when the method indicates that the subject has or is determined to have a disease linked to the expression of POU5F1B.

It is understood that the detection and measurement of the level of transcription of an mRNA encoding POU5F1B, a fragment or variant thereof in a sample obtained can be direct or indirect. For example, the abundance levels of mRNAs can be directly quantitated. Alternatively, the amount of POU5F1B, a fragment or variant thereof can be determined indirectly by measuring abundance levels of cDNAs, amplified RNAs or DNAs, or by measuring quantities or activities of RNAs, or other molecules that are indicative of the expression level of POU5F1B, a fragment or variant thereof. Preferably, the detection and measurement of the level of transcription of an mRNA encoding POU5F1B, a fragment or variant thereof is determined indirectly by measuring abundance levels of cDNAs.

Transcripts (such as mRNAs), amplified RNAs or DNAs (such as cDNAs) can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, and mass spectrometry, as well as any sequencing-based methods known in the art.

In one aspect, microarrays are used to measure the levels of an mRNA encoding POU5F1B, a fragment or variant thereof.

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel. In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm²; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one aspect, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other aspects, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism's genome. In another aspect, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically, each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids. An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., U.S. Pat. No. 5,539,083).

Probes are preferably selected using an algorithm that takes into account binding energies, base composition, sequence complexity, cross-hybridization binding energies, and secondary structure.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA. A second method for making microarrays produces high-density oligonucleotide arrays. Techniques are known for producing arrays containing thousands of oligonucleotides complementary to defined sequences, at defined locations on a surface using photolithographic techniques for synthesis in situ (see, U.S. Pat. Nos. 5,578,832; 5,556,752; and 5,510,270; herein incorporated by reference in their entireties) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 μL or less, more preferably 50 μL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm². The polynucleotide probes are attached to the support covalently at either the 3′ or the 5′ end of the polynucleotide.

POU5F1B polynucleotides which may be measured by microarray analysis can be expressed mRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one aspect, total RNA, mRNAs, or nucleic acids derived therefrom (such as cDNA), are isolated from a sample taken from a patient having cancer or a cancer tissue undergoing surgical and/or pharmacological therapies.

As described above, POU5F1B polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one aspect, POU5F1B polynucleotide molecules from a patient sample are labeled differentially from the corresponding polynucleotide molecules of a reference sample. The reference can comprise mRNAs from a normal biological sample (i.e., control sample, e.g., biopsy from a subject not having a cancer or a cancer tissue undergoing surgical and/or pharmacological therapies) or from a reference biological sample, (e.g., sample from a subject not having a cancer or a cancer tissue undergoing surgical and/or pharmacological therapies). Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer-controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor RNA, mRNA, DNA or cDNA abundance levels at a large number of sites simultaneously.

It is also understood that the methods of the invention also comprise detecting the presence or absence of at least one polypeptide encoded by a nucleic acid sequence of the invention, a fragment or variant thereof, in a biological sample. Detection of said at least one one polypeptide can be direct or indirect and determined by any suitable method known in the art (e.g Western blots, ELISA, HPLC, LC/MS, . . . ). The detection also comprises the determination of one or more post-translation modification of the protein, fragment or variant thereof as described herein.

The present invention further contemplates methods of treating and/or preventing a disease linked to the expression of POU5F1B in a subject in need thereof.

In one aspect of the invention, the method of treating and/or preventing a disease linked to the expression of POU5F1B in a subject in need thereof, comprises administering a therapeutically effective amount of an agent or agent for use of the invention, ii) a gene delivery vector of the invention, iii) a host cell of the invention, or iv) a pharmaceutical composition of the invention.

In one aspect of the invention, the method of treating and/or preventing a disease linked to the expression of POU5F1B in a subject in need thereof, comprises modifying a host cell, and reintroducing the host cell (e.g. single cell or population of cells) into the subject in need thereof.

Preferably, a biopsy or other tissue or biological fluid sample comprising the single cell or the population of cells may be necessary. Cells such as fibroblast cells or stem cells that can be generated directly from adult cells, such as iPSCs, are particularly preferred in this regard.

A gene delivery vector (e.g. plasmid or vector) comprising a nucleic acid encoding an agent or agent for use of the invention or at least one acid nucleic of the invention can be introduced to host cell via one or more methods known in the art. These one or more methods include, without limitation, microinjection, electroporation, calcium phosphate-mediated transfection, cationic transfection, liposome transfection, dendrimer transfection, heat shock transfection, nucleofection transfection, magnetofection, lipofection, optical transfection, proprietary agent-enhanced uptake of nucleic acids, and delivery via liposomes, immunoliposomes, virosomes, or artificial virions.

The host cell (e.g. single cell or population of cells) of the invention is then reintroduced into the subject in need thereof by any route of administration and/or delivery methods known in the art, as described herein.

The invention also contemplates kits for performing a method according to the invention or for the treatment and/or prevention of a disease linked to the expression of POU5F1B. In one aspect of the invention, the kit comprises a pharmaceutical composition comprising an agent of the invention.

In a further aspect, the invention contemplates a kit for the treatment and/or prevention of a disease linked to the expression of POU5F1B comprising a host cell of the invention.

The kits of the invention may also comprise a container and a label or package insert on or associated with the container. Suitable containers include, for example, bottles, vials, syringes, etc. The containers may be formed from a variety of materials such as glass or plastic. The container holds a composition which is effective for treating the disease of disorder of the invention and may have a sterile access port (for example the container may be an intravenous solution bag or a vial having a stopper pierceable by a hypodermic injection needle). Alternatively, or additionally, the kits may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The label or package insert may comprise instructions for use thereof. Instructions included may be affixed to packaging material or may be included as a package insert. While the instructions are typically written or printed materials they are not limited to such. Any medium capable of storing such instructions and communicating them to an end user is contemplated by this disclosure.

In some aspects, the kit comprises, alternatively or additionally, one or more oligonucleotide probes (e.g. fixed on a solid support or surface as described herein) or primers described herein.

Further contemplated in the present invention are nucleic acids encoding an siRNA, an shRNA, an snRNA, a siRNA capable of interfering the expression of short hairpin (sh), a piRNA, or a nucleic acid including an antisense oligonucleotide (e.g. ASOs, modified ASOs such as GapmeRs, . . . ) of the invention.

The invention also contemplates the use of an agent of the invention in the manufacture of a medicament for treating and/or preventing a disease linked to the expression of POU5F1B.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

Sequences

Human POU5F1 (SEQ ID No. 1) (see FIG. 1A)
Human POU5F1B (SEQ ID No. 2) (see FIG. 1A)
Gorilla POU5F1B (SEQ ID No. 3) (see FIG. 1A)
Orangutan POU5F1B (SEQ ID No. 4) (see FIG. 1A)
shRNA1_POU5F1B (SEQ ID No. 5)
5′-TGCTGTTGACAGTGAGCGCACAGGTGATTATGATTTAAAGTAG
TGAAGCCACAGATGTACTTTAAATCATAATCACCTGTGTGCCTACT
GCCTCGGA-3′
shRNA2_POU5F1B (SEQ ID No. 6)
5′-TGCTGTTGACAGTGAGCGCACATTCAGTCAACATTTAATGTAG
TGAAGCCACAGATGTACATTAAATGTTGACTGAATGTGTGCCTACT
GCCTCGGA-3′)
siRNA_ZDHHC17 (SEQ ID NO. 7)
5′-CAGTACCTGTTTGATACGAAA-3′
POU5F1B primer Fw (SEQ ID No. 8)
5′-CACCATGGCGGGACACCTGGCTTCGGATTTC-3′,
POU5F1B primer Rev (SEQ ID No. 9)
5′-GTTTGAATGCATGGGAGAGCCCAG-3′
LTR66-g1 (SEQ ID No. 10)
5′-TCACATCATTCTCACCACTCTGG-3′
LTR66-g2 (SEQ ID No. 11)
5′-GGAGCAGTCTCCTGAAGCTTTGG-3′.
enh-g1 (SEQ ID No. 12)
5′-GACGATGAGGGTATTAACTCTGG-3′
enh-g2 (SEQ ID No. 13)
5′-GGTAATATGTTTGGGCCTGTAGG-3′.
POU5F1B_g1 (SEQ ID No. 14)
5′-CTAGGTCATAAAGAACGCAG-3′
POU5F1B_g2 (SEQ ID No. 15)
5′-ATGCCGTTAGCGGTCAAAAG-3′.
Chimpanzee POU5F1B (SEQ ID No. 16)
(see FIG. 1A)

| single_guide_RNA_ | | |
|---|---|---|
| sequence (5′>3′) | Gene | SEQ |
| targeting MET gene | Symbol | ID No. |
| AGCTGTGGCAGCGTCAACAG | MET | 17 |
| CCGATCGCACACATTTGTCG | MET | 18 |
| CTCACTGATATCGAATGCAA | MET | 19 |
| TACTGTATTGTGTTGTCCCG | MET | 20 |
| TGAGATCAAAGTATTTGGAA | MET | 21 |
| TGATGTCCCAAGATTAGCTA | MET | 22 |
| TGCGTATGTCAGCAAGCCTG | MET | 23 |
| single_guide_RNA_ | | |
| sequence (5′>3′) | Gene | SEQ |
| targeting CD47 gene | Symbol | ID No. |
| ACAGGAGTATAGCAAAAATT | CD47 | 24 |
| AGTGATGCTGTCTCACACAC | CD47 | 25 |
| ATCGAGCTAAAATATCGTGT | CD47 | 26 |
| CTACTGAAGTATACGTAAAG | CD47 | 27 |
| CTTACCTGGGACGAAAAGAA | CD47 | 28 |
| GCACTTAAATATAGATCCGG | CD47 | 29 |
| TTGCACTACTAAAGTCAGTG | CD47 | 30 |

### EXAMPLES

### Example 1

### Material and Methods

Cell culture. SW480 (ATCC) were cultured in L15 medium (Sigma), supplemented with 10% FCS. For lentiviral vector production, 293T cells were cultured in DMEM supplemented with 10% FBS with 100 IU ml⁻¹ penicillin, 100 ug ml⁻¹ streptomycin, and 26 μg ml⁻¹ glutamine (Corning 30-009-CI) at 37° C. in a humidified atmosphere of 5% CO₂. All cells tested negative for mycoplasma.

Overexpression and shRNA vectors. For the POU5F1B-expressing vector, genomic DNA from DLD1 cells (ATCC® CCL-221™) was PCR amplified with the POU5F1B primers 5′-CACCATGGCGGGACACCTGGCTTCGGATTTC-3′ (SEQ ID No. 8), 5′-GTTTGAATGCATGGGAGAGCCCAG-3′ (SEQ ID No. 9), cloned into a pENTR TOPO donor vector (Thermo Fisher), that was recombined with the doxycycline-inducible lentiviral destination vector pSin-TRE-3×HA-puro. Human codon optimized versions of our POU5F1B-expressing vector and the K135E, K182T and S95G mutants were performed by GeneScript Biotech.

Lentivirus production and stable gene expression. Lentiviral particles produced as described at http://tronolab.epfl.ch were used to transduce SW480 cells in 6-well plates at multiplicity of infection 1, before selection in 1 μg ml⁻¹ puromycin for 7 days.

Immunofluorescence. Cell lines were plated on glass coverslips into 24-well plates and cultured for 3 days up to 70% confluence in 500 ng ml⁻¹ doxycycline-containing medium. When needed, cells were treated with TAK-243, inhibitor of the ubiquitin activating enzyme, at 1 μM for 5 hours. Growth medium was refreshed (1 ml) prior a 15 min fixation with 1 ml 8% paraformaldehyde (PFA) added dropwise. Cells were washed three times with PBS, permeabilized with 0.1% saponin PBS for 20 min, and blocked with 1% BSA 0.1% saponin PBS for 30 min before incubation with anti-HA (bioLegend, Covance catalog #MMS-101P, 1:1,000) antibody in 1% BSA 0.1% saponin PBS 2 h at room temperature (RT) under agitation. Samples were washed three times with PBS and incubated with Alexa 647-conjugated (A647) anti-mouse antibody (1:1,000) in 1% BSA 0.1% saponin PBS for 40 min at RT. Three final washes were performed before mounting the slides in Vectashield with DAPI (Vector Laboratories). Images were acquired on a ZEISS LSM 700 confocal microscopy and analyzed with Fiji software.

Isolation of detergent-resistant membranes (DRMs). Approximately 1×10⁷ cells were resuspended in 0.5 ml cold TNE buffer (25 mMTris-HCl, pH 7.5, 150 mM NaCl, 5 mM EDTA, and 1% Triton X-100; Surfact-Amps, ThermoFisher) with a tablet of protease inhibitors (Roche). Membranes were solubilized in a rotating wheel at 4° C. for 30 min. DRMs were isolated using an Optiprep™ gradient ⁷²: the cell lysate was adjusted to 40% Optiprep™, loaded at the bottom of a TLS. 55 Beckman tube, overlaid with 600 μl of 30% Optiprep™ and 600 μl of TNE, and centrifuged for 1.5 h at 259,000 g at 4° C. Six fractions of 400 μl were collected from top to bottom. DRMs were found in fraction 2. Equal volumes from each fraction were analyzed by SDS-PAGE and western blot analysis using HRP-conjugated anti-HA, caveolin 1 (Santa Cruz sc-894, 1:500) and transferrin receptor (ThermoFisher 13-6800, 1:1,000) antibodies.

In vitro proliferation assays. Cell proliferation was determined by the 3-(4,5-dimethylthiazol-2-yl)-2,5-diphenyltetrazolium bromide (MTT) assay for 5-6 days, staining the cells with 5 mg ml-1 MTT for 3 h, removing the medium, adding DMSO to dissolve the crystals and measuring absorbance at 560 nm.

Immunoprecipitation. Cells were washed three times PBS, lysed 30 min at 4° C. in the following Buffer (0.5% Nonidet P-40, 500 mM Tris pH 7.4, 20 mM EDTA, 10 mM NaF, 2 mM benzamidin and protease inhibitor cocktail (Roche)), and centrifuged 3 min at 5000 rpm. Supernatants were subjected to preclearing with mouse IgG-agarose beads (Sigma A0919) prior immunoprecipitation reaction. Supernatants were incubated overnight with anti HA-agarose beads (Sigma A2095). Similar elution volumes and protein quantities, for the IPs and the inputs, respectively, were submitted to SDS-PAGE and analyzed by immunoblotting using anti-ubiquitin P4D1 (Santa Cruz Biotechnology sc-8017, 1:1,000), HRP-conjugated anti-HA (clone 3F10 Roche 12013819001, 1:1,000), anti-phosphoserine (P5747, Sigma) and HRP-conjugated anti-mouse antibody.

Acyl-RAC capture assay. Protein S-palmitoylation was assessed by the Acyl-RAC assay as previously described (Werno and Chamberlain, 2015) with some modifications. Cells or supernatants were lysed in 400 ml buffer (0.5% Triton-X100, 25 mM HEPES, 25 mM NaCl, 1 mM EDTA, pH 7.4, and protease inhibitor cocktail). Cell lysis were incubated 30 min at RT with 10 mM TCEP. Then, 200 ml of blocking buffer (100 mM HEPES, 1 mM EDTA, 87.5 mM SDS, and 1.5% [v/v] methyl methanethiosulfonate (MMTS)) was added to the lysates and incubated for 4 h at 40° C. to block free the SH groups with MMTS. Proteins were acetone precipitated and resuspended in buffer (100 mM HEPES, 1 mM EDTA, 35 mM SDS). For treatment with hydroxylamine (NH₂OH) and capture by Thiopropyl Sepharose beads, 2 M of hydroxylamine was added together with the beads (previously activated for 15 min with water) to a final concentration of 0.5 M of hydroxylamine and 10% (w/v) beads. As a negative control, 2 M Tris was used instead of hydroxylamine. These samples were then incubated overnight at room temperature on a rotating wheel. After washes, the proteins were eluted from the beads by incubation in 40 ml SDS sample buffer with β-mercaptoethanol for 5 min at 95° C. Samples were loaded in SDS-PAGE gels and analyzed by Western blot. A fraction of the cell lysate (total cell extract) was saved as the input.

ZDHHC genes silencing. All human ZDHHC genes were silenced for 72 h in SW480 cells by Lipofectamine-RNAiMAX-mediated transfection of siRNAs (Qiagen), the sequences of which were previously published (Lakkaraju, A. K. et al. 2012, EMBO J. 31, 1823-1835). Silencing efficiency was checked by qPCR.

Dose response assays. POU5F1B-DCK-, DCK-overexpressing, and wild type SW480 cells were seeded in 96-well plates at a cell density of 2,000 cells/well in 100 μl of media. At the same time of cell seeding, 100 μM Capivasertib, OXA-06 and Laurocapram dissolved in DMSO were diluted in medium and 20 μl were dispensed in the 96-wells to achieve final concentrations of 0.39, 0.78, 1.56, 3.12, 6.25, 12.5 and 25 μM, keeping the final concentration of DMSO at 0.2% in each well. The next day, 500 μM BVdU (Brivudine H27853, Thermo Fisher) was added in all wells except for non-treated ones. Plate reading was done at 96 h and 120 h post BvDU treatment by measuring fluorescence after incubating the cells with PrestoBlue™ (A13262, Invitrogen) for 30 min.

Radiolabelling experiment to determine POU5F1B half-life. POU5F1B-overexpressing SW480 cells treated for 24 hours with capivasertib (20 μM), oxa-06 (20 μM) and Y-27632 (20 μM) ROCK inhibitors, and 3 hours with MG132 (10 μM) proteasome inhibitor, were washed with methionine/cysteine-free medium, incubated for a 20-min pulse at 37° C. with 50 μCi/ml [35S]methionine/cysteine (Hartman Analytics), washed, and further incubated for different times at 37° C. in complete medium with a 10-fold excess of nonradioactive methionine and cysteine. POU5F1B protein was HA-immunoprecipitated, eluted and loaded in SDS-PAGE gels, and autoradiographies were quantified using the Typhoon Imager (Image QuantTool, GE healthcare).

### Results

The Inventors demonstrated that POU5F1B is ubiquitinylated, and this modification is essential for its oncogenic function. They further showed that inhibiting the El ubiquitin-activating enzyme, responsible for the first step of the ubiquitinylation reaction, with TAK-243, triggers the re-localization of POU5F1B to the nucleus, which impairs its function. They also found that POU5F1B can be immunoprecipitated with two E3 ubiquitin ligases, UBR5 and RNF213, and is found in close proximity to a third one, TRIM21. They finally observed that POU5F1B is stabilized by treatment with MG132, indicating that it is normally degraded in the proteasome, as expected from a poly-ubiquitinylated protein. All of these findings suggest that any alteration/targeting of the ubiquitinylation chain reaction is likely to alter POU5F1B function.

### Example 2

We performed a positive selection screen using an sgRNA druggable library as described in Koduri et al 2020 Sci Advances (DOI: 10.1126/sciadv.abd6263). Briefly:

sgRNA druggable library. We used the CP1080 sgRNA library (provided by David Root-Broad Institute) that was custom-designed to target cancer-relevant druggable genes. It consisted of 5566 sgRNAs targeting 788 genes (7 sgRNAs targeting each gene) and 300 nontargeting sgRNAs as controls.

Positive selection CRISPR-Cas9 BVdU resistance screen. SW480 cells were transduced with pLX304 POU5F1B-DCK-V5-IRES-GFP or pLX304 DCK-V5-IRES-GFP lentivectors and subsequently maintained in blasticidin selection. Blasticidin-resistant cells were sorted for GFP expression (top 1%) three times by FACS. Sorted cells were transduced with pL Vx Cas9-flag-IRES-hygro lentivectors and placed under hygromycinB selection. Functionality of POU5F1B-DCK or DCK alone was determined using BVdU sensitivity experiment. POU5F1B-DCK and DCK cells expressing Cas9 were expanded and then counted. For each line, 2.2×107 cells (~4000 cells per sgRNA) were transduced at a multiplicity of infection (MOI) of ~0.3 with the sgRNA druggable lentivector library (CP1080) described above. The cells were then cultured for 48 hours before being placed under puromycin (3 u g/ml) selection for 4 days.

On day 0, the puromycin resistant cells were counted. For each condition, a total of 2×107 cells were collected and washed in PBS, and the cell pellets were frozen for genomic DNA isolation for the initial time point before BVdU selection, and 2×107 cells were treated with BVdU doses corresponding to their sensitivity, i.e. 100 μ M and 200 μ M for the DCK-Cas9-CP1080 cells and 400 μ M and 800 μ M for the POU5F1B-DCK-Cas9-CP180 cells. On days 4, 11, 17 and 23 cells were counted and replated in complete media containing BVdU.

On day 23, POU5F1B-DCK-Cas9-CP1080 400 μ M cells were split in 2, half of the culture was kept in 400 μ M BVdU and the rest was treated with 600 μ M BVdU to increase the selection pressure.

On day 33, a total of 10×106 cells from each arm of the screen were resuspended in complete media containing their correponding BvdU concentration. If available, the remaining cells were centrifuged and washed in PBS, and the cell pellets were frozen. Thus, at least 1000 cells per sgRNA were maintained under BVdU selection.

On day 37, all remaining cells were collected, counted, and divided in aliquots of 6×106 cells (which corresponds to 1000 cells per sgRNA) and washed in PBS. The cell pellets were frozen for genomic DNA isolation for the final time point after BVdU selection. Following completion of the screen, genomic DNA was isolated using a Machery Nagel Nucleospin Blood Kit_XL (reference 740950.1) according to the manufacturer's protocol. Raw Illumina reads were normalized between samples using log 2 [(sgRNA reads/total reads for sample)×1×106+1]. The initial time point data (day 0) were then subtracted from the end time point after BVdU selection (day 37) to determine the relative enrichment of each individual sgRNA after BVdU treatment using hypergeometric analysis and the STARS algorithm. A q value cutoff of <0.25 was used to call hits. The averaged data from 3× POU5F1B-DCK and 2× DCK biological replicates were used for all analyses.

### Results

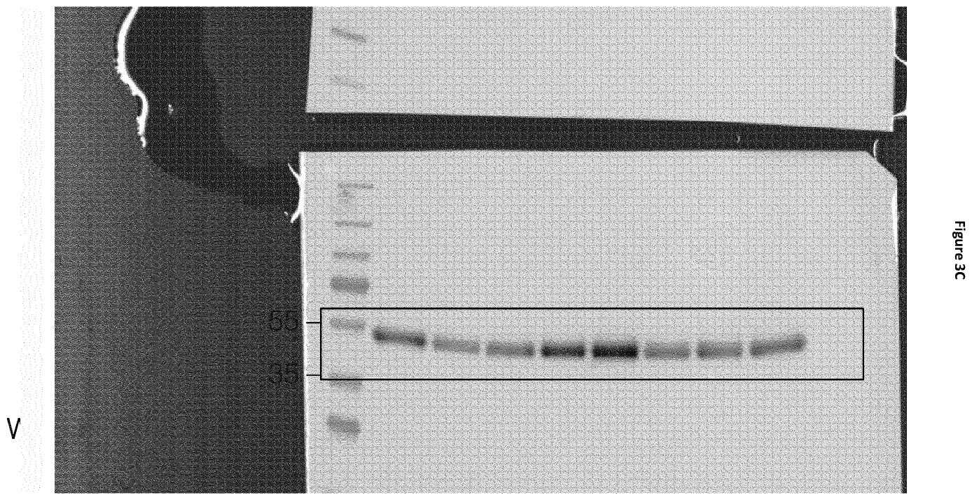

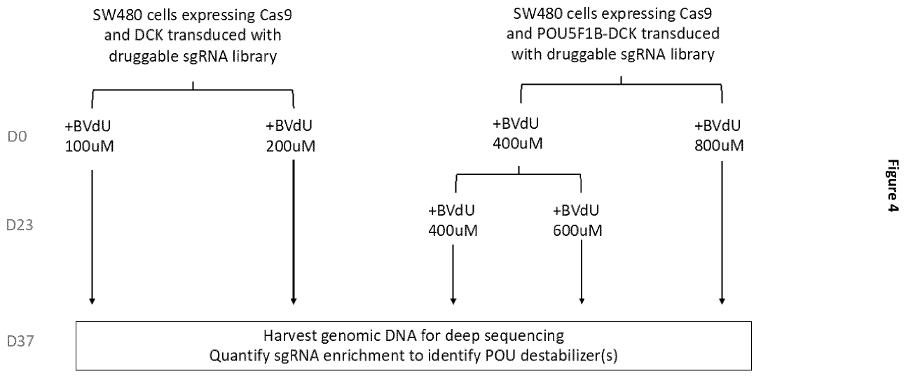

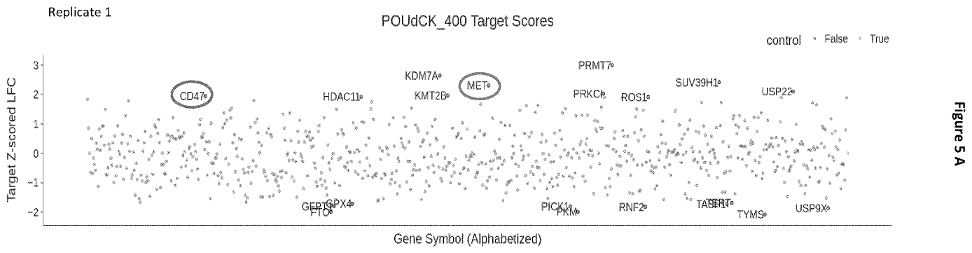

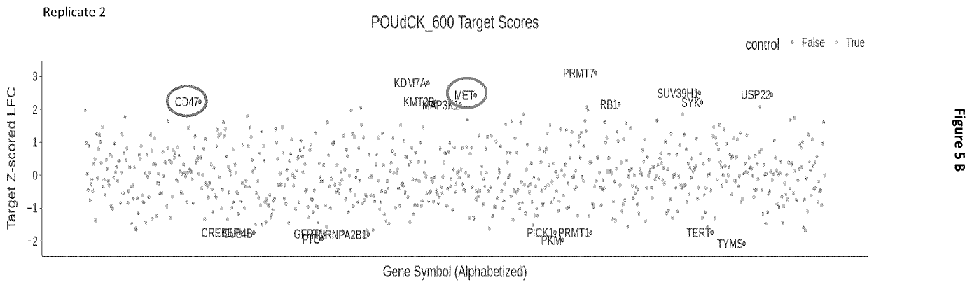

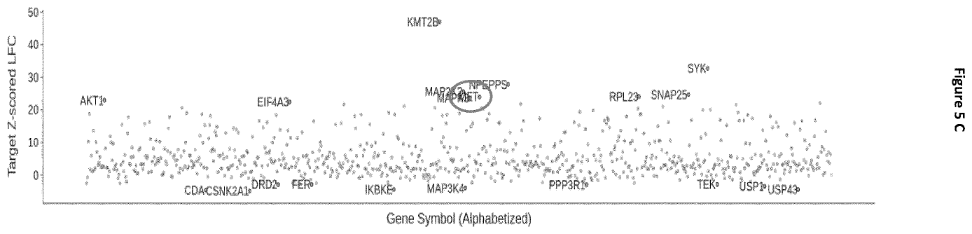

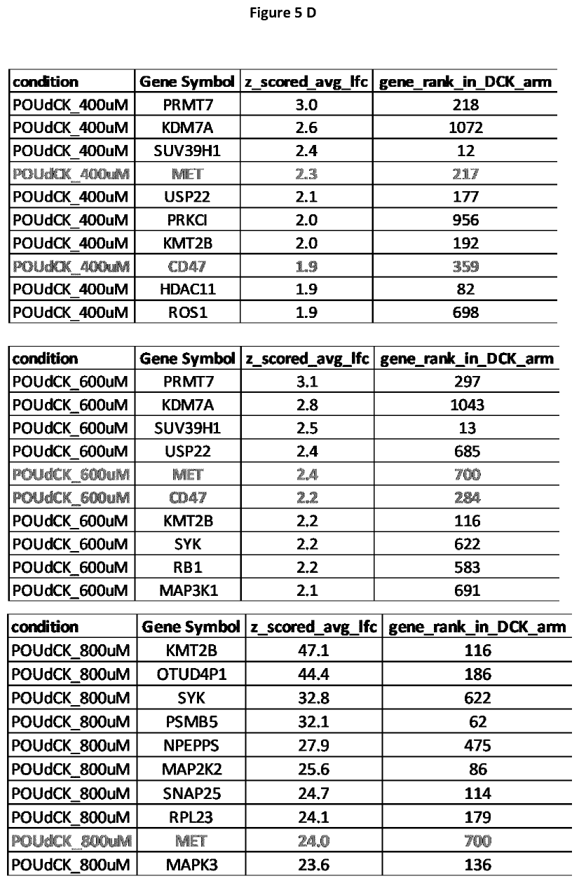

Following completion of the screen (FIG. 4), we determined sgRNA abundance by next-generation sequencing of genomic DNA extracted from 3× POU5F1B-DCK and 2× DCK biological replicates and analyzed relative enrichment of sgRNAs compared to the time point before BVdU treatment. We identified multiple sgRNAs against MET and CD47 that were markedly enriched in both BvDU concentrations in the POU5F1B-DCK cells but not the DCK cells (FIG. 5).

1. A method for treating and/or preventing a disease linked to the expression of POU5F1B in a subject in need thereof, the method comprising administering a therapeutically effective amount of an agent that modulates the expression and/or activity of i) the POU5F1B protein, a fragment or variant thereof, ii) an mRNA encoding POU5F1B, a fragment or variant thereof, and/or iii) the POU5F1B gene.

2. The method of claim 1, wherein the disease linked to the expression of POU5F1B comprises cancer and/or cancer metastasis.

3. The method of claim 1, wherein the agent comprises a chemical agent, an oligonucleotide, an antibody or an antigen-binding fragment thereof, a peptide, or a combination thereof.

4. The method of claim 1, wherein the agent modulates the expression and/or activity of the POU5F1B protein, fragment or variant thereof, by i) inhibiting or impairing, directly or indirectly, at least one post-translational modification of the POU5F1B protein, fragment or variant thereof, ii) inducing selective intracellular proteolysis of the POU5F1B protein, fragment or variant thereof, or iii) inhibiting or impairing, directly or indirectly, the stability of the POU5F1B protein, fragment or variant thereof.

5. The method of claim 4, wherein the at least one post-translational modification comprises ubiquitylation, palmitoylation, or phosphorylation of the POU5F1B protein, fragment or variant thereof.

6. The method of claim 4, wherein the agent inhibiting or impairing at least one post-translational modification targets an El ubiquitin activating enzyme, an E3 ubiquitin ligase, a palmitoyltransferase or a kinase, or a combination thereof.

7. The method of claim 6, wherein the E1 ubiquitin activating enzyme comprises UBA1, UBA2, UBA3, UBA5, UBA6, UBA7, ATG7, NAE1, or SAE1.

8. The method of claim 6, wherein the palmitoyltransferase comprises ZDHHC-17.

9. The method of claim 6, wherein the kinase comprises a ROCK kinase.

10. The method of claim 4, wherein the agent inhibiting or impairing, the stability of the POU5F1B protein, fragment or variant thereof targets MET or CD47, or a combination thereof.

11. The method of claim 3, wherein said agent is an oligonucleotide comprising an siRNA, an shRNA, an snRNA, an siRNA capable of interfering the expression of short hairpin (sh), a guide RNA, a piRNA, an antisense oligonucleotide, or a combination of one or more thereof.

12. The method of claim 8, wherein the agent targeting palmitoyltransferase ZDHHC17 comprises an siRNA, an shRNA, an snRNA, or an siRNA capable of interfering the expression of short hairpin (sh).

13. The method of claim 4, wherein the agent inducing selective intracellular proteolysis of the POU5F1B protein, fragment or variant thereof is a chemical compound comprising a proteolysis targeting chimera (PROTAC) protein degrader, a ROCK kinase inhibitors, a small-molecule chemical modulator of a ubiquitinating enzyme upstream of or on a proteasome, or a combination of one or more thereof.

14. The method of claim 13, wherein the ROCK kinase inhibitor comprises AZD5363, OXA-06, Y-27632, or a combination of one or more thereof.

15. The method of claim 10, wherein the agent targeting MET is an sgRNA comprising SEQ ID NO: 17, SEQ ID NO: 18, SEQ ID NO: 19, SEQ ID NO: 20, SEQ ID NO: 21, SEQ ID NO: 22, SEQ ID NO: 23, or a sequence sharing at least 80%, 85%, 90%, 95%, 98% or 99% sequence identity therefrom.

16. The method of claim 10, wherein the agent targeting CD47 is an sgRNA comprising SEQ ID NO: 24, SEQ ID NO: 25, SEQ ID NO: 26, SEQ ID NO: 27, SEQ ID NO: 28, SEQ ID NO: 29, SEQ ID NO: 30, or a sequence sharing at least 80%, 85%, 90%, 95%, 98% or 99% sequence identity therefrom.

17. The method of claim 1, wherein the agent targets i) one or more transposable elements that serve as promoter of the POU5F1B gene, ii) one or more enhancer of the POU5F1B gene or iii) one or more flanking regions of the POU5F1B gene.

18. The method of claim 17, wherein the one or more transposable elements that serve as promoter of the POU5F1B gene comprises AluJr4, HAL1, AluSg7, L1PA16, AluSx, LaPA8, L1PA5, L1PA8, L1PA7, AluJr, L1PA7, MIR, L2a, L2b, L2c, MIRb, LTR16A, THE1D, MLT2B4, ERVL-B4-int, LTR12, MIR3, L1PA3, L1PA2, L2, LTR33, DCP2, YIPF4, LTR66, L2d2, a variant thereof, a fragment thereof, or a combination thereof.

19. The method of claim 17, wherein the one or more enhancer of the POU5F1B gene is located within locations hg19 chr8: 128,409,691-128,415,139, and/or hg19 chr8: 128,402,759-128,405,287.

20. The method of claim 17, wherein the agent targeting the one or more enhancer of the POU5F1B gene comprises SEQ ID NO: 12 and/or SEQ ID NO: 13, or a sequence sharing at least 80%, 85%, 90%, 95%, 98% or 99% sequence identity therefrom.

21. The method of claim 2, wherein the cancer is a solid tumor cancer comprising colorectal cancer (COAD and READ), cervical squamous cell carcinoma and endocervical adenocarcinoma (CESC), bladder cancer (BLCA), breast cancer (BRCA), uterine corpus endometrial carcinoma (UCEC), ovarian cancer (OV), esophageal cancer (ESCA), stomach cancer (STAD), kidney cancer (KICH, KIRC, and KIRP), lung squamous cell carcinoma (LUSC), lung adenocarcinoma (LUAD), prostate cancer (PRAD), thyroid carcinoma (THCA), thymoma (THYM), or a combination of one of more thereof.

22. A gene delivery vector comprising a nucleic acid encoding an agent that modulates the expression and/or activity of i) the POU5F1B protein, a fragment or variant thereof, ii) an mRNA encoding POU5F1B, a fragment or variant thereof, and/or iii) the POU5F1B gene,
wherein the agent is an oligonucleotide comprising an siRNA, an shRNA, an snRNA, an siRNA capable of interfering the expression of short hairpin (sh), a guide RNA, a piRNA, an antisense oligonucleotide, or a combination of one or more thereof.

23. A host cell comprising, or modified by the introduction of:
a) a nucleic acid encoding an agent that modulates the expression and/or activity of i) the POU5F1B protein, a fragment or variant thereof, ii) an mRNA encoding POU5F1B, a fragment or variant thereof, and/or iii) the POU5F1B gene, wherein the agent is an oligonucleotide comprising an siRNA, an shRNA, an snRNA, an siRNA capable of interfering the expression of short hairpin (sh), a guide RNA, a piRNA, an antisense oligonucleotide, or a combination of one or more thereof; or
b) the gene delivery vector of claim 22.

24. A pharmaceutical composition comprising a therapeutically effective amount of:
a) an agent that modulates the expression and/or activity of i) the POU5F1B protein, a fragment or variant thereof, ii) an mRNA encoding POU5F1B, a fragment or variant thereof, and/or iii) the POU5F1B gene; or
b) a gene delivery vector of claim 22; or
c) a host cell comprising the gene delivery vector of claim 22;
and a pharmaceutically acceptable carrier and/or diluent.

25. A method for diagnosing a disease linked to the expression of POU5F1B in a biological sample of a subject, the method comprising:
i) detecting, directly or indirectly, the expression and/or activity of the POU5F1B protein, a fragment or variant thereof, or
ii) detecting, directly or indirectly, the presence of an mRNA encoding POU5F1B, a fragment or variant thereof,
wherein the expression and/or activity of the POU5F1B protein, a fragment or variant thereof or the presence of an mRNA encoding the POU5F1B protein, a fragment or variant thereof indicates that the subject has or is determined to have a disease linked to the expression of POU5F1B.

26. (canceled)

27. A method of treating and/or preventing a disease linked to the expression of POU5F1B in a subject in need thereof, the method comprising
modifying a host cell by the introduction of:
a) a nucleic acid encoding an agent that modulates the expression and/or activity of i) the POU5F1B protein, a fragment or variant thereof, ii) an mRNA encoding POU5F1B, a fragment or variant thereof, and/or iii) the POU5F1B gene, wherein the agent is an oligonucleotide comprising an siRNA, an shRNA, an snRNA, an siRNA capable of interfering the expression of short hairpin (sh), a guide RNA, a piRNA, an antisense oligonucleotide, or a combination of one or more thereof; or
b) the gene delivery vector of claim 22;
and reintroducing the host cell into the subject in need thereof.


SEQUENCE LISTING (30 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 359 aa, Homo sp.
MAGHLASDFAFSPPPGGGGDGPWGAEPGWVDPLTWLSFQGPPGGPGIGPGVGPGSEVWGIPPCPPPYELCGGMAYCGPQVGVGLVPQGGLETSQPESEAGVGVESNSNGASPEPCTVPPGAVKLEKEKLEQNPEKSQDIKALQKELEQFAKLLKQKRITLGYTQADVGLILGVLFGKVFSQKTICRFEALQLSFKNMCKLRPLLQKWVEEADNNENLQEICKAETLMQARKRKRTSIENRVRGNLENLFLQCPKPTLQISHIAQQLGLEKDVVRVWFCNRRQKGKRSSSDYAQREDFEAAGSPFSGGPVSFPPAPGPHFGTPGYGSPHFTALYSSVPFPEGEVFPPVSVITLGSPMHSN

SEQ ID NO: 3 - protein, 358 aa, Gorilla gorilla
MAGHLASDFAFSPPPGGGGDGPGGTEPGWVDPLTWLSFQGPPGGPGIGPGAGPGSEVWGIPPCPPPYEFCRGMAYCGPQVGVGLVPQGGLETSQPEGEAGVGVESNSNGASPEPCTVPPGAVKLEKEKLEQNPESQDIKALQKELEQFAKLLKQKRITLGYTQADVGLILGVLFGKVFSQTTICCFEALQLSFKNMCKLRPLLQKWVEEADNNENLQEICKAESLMQARKRKRTSIENRVRGNLENLFLQCPKPTLQISHIAQQLGLEKDVVRVWFCNRRQKGKRSSSDYAQREDFEAAGSPFSGGPVSFPPAPGPHFGTPGYGSPHFTALYSSVPFPEGEVFPPVSVITLGSPMHSN

SEQ ID NO: 4 - protein, 358 aa, Pongo sp.; identical to SEQ ID NO: 3

SEQ ID NO: 5 - DNA, 97 bp, synthetic construct
  source 1..97: mol_type = other DNA
tgctgttgacagtgagcgcacaggtgattatgatttaaagtagtgaagccacagatgtactttaaatcataatcacctgtgtgcctactgcctcgga

SEQ ID NO: 6 - DNA, 97 bp, synthetic construct
  source 1..97: mol_type = other DNA
tgctgttgacagtgagcgcacattcagtcaacatttaatgtagtgaagccacagatgtacattaaatgttgactgaatgtgtgcctactgcctcgga

SEQ ID NO: 7 - DNA, 21 bp, synthetic construct
  source 1..21: mol_type = other DNA
cagtacctgtttgatacgaaa

SEQ ID NO: 8 - DNA, 31 bp, synthetic construct
  source 1..31: mol_type = other DNA
caccatggcgggacacctggcttcggatttc

SEQ ID NO: 9 - DNA, 24 bp, synthetic construct
  source 1..24: mol_type = other DNA
gtttgaatgcatgggagagcccag

SEQ ID NO: 10 - DNA, 23 bp, synthetic construct
  source 1..23: mol_type = other DNA
tcacatcattctcaccactctgg

SEQ ID NO: 11 - DNA, 23 bp, synthetic construct
  source 1..23: mol_type = other DNA
ggagcagtctcctgaagctttgg

SEQ ID NO: 12 - DNA, 23 bp, synthetic construct
  source 1..23: mol_type = other DNA
gacgatgagggtattaactctgg

SEQ ID NO: 13 - DNA, 23 bp, synthetic construct
  source 1..23: mol_type = other DNA
ggtaatatgtttgggcctgtagg

SEQ ID NO: 14 - DNA, 20 bp, synthetic construct
  source 1..20: mol_type = other DNA
ctaggtcataaagaacgcag

SEQ ID NO: 15 - DNA, 20 bp, synthetic construct
  source 1..20: mol_type = other DNA
atgccgttagcggtcaaaag

SEQ ID NO: 16 - protein, 359 aa, Pan troglodytes
MAGHLASDFAFSPPPGGEGDGPGGPEPGWVDPLTWLRFQGPPGGPGIGPGVGPGSEVWGIPPCPPPYEFCGGMAYCGPQVGAGLVPQGGLETSQPEGEAGVGVESNSDGASPEPCTVPPGAVKLEKEKLEQNPEESQDIKALQKELEQFAKLLKQKRITLGYTQADVGLILGVLFGKVFSQTTICRFEALQLSFKNMCKLRPLLQKWVEETDNNENLQEICKAETLMQARKRKRTSIENRVRGNLENLFLQCPKPTLQISHIAQQLGLEKDVVRVWFCNRRQKGKRSSSDYEQREDFEAAGSPFSGGPVSFPLAPGPHFGTPGYGSPHFTALYSSVPFPEGEVFPPVSVITLGSPMHLN

SEQ ID NO: 17 - DNA, 20 bp, synthetic construct
  source 1..20: mol_type = other DNA
agctgtggcagcgtcaacag

SEQ ID NO: 18 - DNA, 20 bp, synthetic construct
  source 1..20: mol_type = other DNA
ccgatcgcacacatttgtcg

SEQ ID NO: 19 - DNA, 20 bp, synthetic construct
  source 1..20: mol_type = other DNA
ctcactgatatcgaatgcaa

SEQ ID NO: 20 - DNA, 20 bp, synthetic construct
  source 1..20: mol_type = other DNA
tactgtattgtgttgtcccg

SEQ ID NO: 21 - DNA, 20 bp, synthetic construct
  source 1..20: mol_type = other DNA
tgagatcaaagtatttggaa

SEQ ID NO: 22 - DNA, 20 bp, synthetic construct
  source 1..20: mol_type = other DNA
tgatgtcccaagattagcta

SEQ ID NO: 23 - DNA, 20 bp, synthetic construct
  source 1..20: mol_type = other DNA
tgcgtatgtcagcaagcctg

SEQ ID NO: 24 - DNA, 20 bp, synthetic construct
  source 1..20: mol_type = other DNA
acaggagtatagcaaaaatt

SEQ ID NO: 25 - DNA, 20 bp, synthetic construct
  source 1..20: mol_type = other DNA
agtgatgctgtctcacacac

SEQ ID NO: 26 - DNA, 20 bp, synthetic construct
  source 1..20: mol_type = other DNA
atcgagctaaaatatcgtgt

SEQ ID NO: 27 - DNA, 20 bp, synthetic construct
  source 1..20: mol_type = other DNA
ctactgaagtatacgtaaag

SEQ ID NO: 28 - DNA, 20 bp, synthetic construct
  source 1..20: mol_type = other DNA
cttacctgggacgaaaagaa

SEQ ID NO: 29 - DNA, 20 bp, synthetic construct
  source 1..20: mol_type = other DNA
gcacttaaatatagatccgg

SEQ ID NO: 30 - DNA, 20 bp, synthetic construct
  source 1..20: mol_type = other DNA
ttgcactactaaagtcagtg